(19) United States
(12) Patent Application Publication
(10) Pub. No.: US 2014/0031949 A1
(43) Pub. Date: Jan. 30, 2014

(54) Ceramic antibacterial

(71) Applicant: Louis A. Serafin, JR., Lakeport, MI (US)

(72) Inventor: Louis A. Serafin, JR., Lakeport, MI (US)

(73) Assignee: Signal Medical Corporation, Marysville, MI (US)

(21) Appl. No.: 13/987,014

(22) Filed: Jun. 25, 2013

Related U.S. Application Data
(60) Provisional application No. 61/690,473, filed on Jun. 27, 2012.

(51) Int. Cl. A61L 27/54

(52) U.S. Cl. CPC A61L 27/54
USPC 623/23.56

(57) ABSTRACT

Bacterial growth or infection, especially with respect to an implant and/or tissue about the implant, can be ameliorated, controlled or eliminated by identifying a potential for or a presence of bacterial growth or infection with respect to tissue about an implant proposed for implantation; selecting as the implant a ceramic implant; and implanting the ceramic implant under conditions such that bacterial growth or infection, especially with respect to the implanted ceramic implant and/or tissue about the implanted ceramic implant, is ameliorated, controlled or eliminated at least in part from the presence of the implanted ceramic implant. Among ceramics that may be employed is a magnesium oxide stabilized transformationally toughened zirconia. The ceramic implant may be a prosthesis, which may be a load-bearing joint replacement implant or implant component.

This claims the benefits under 35 USC 119(e) of provisional patent application No. US 61/690,473 filed on Jun. 27, 2012 A.D. The specification of that application in its entirety, to include its drawings, is incorporated herein by reference.

### FIELD AND PURVIEW OF THE INVENTION

This concerns treatment of bacterial growth, especially with respect to tissue about an implant prosthesis, through employment of a ceramic body. The ceramic body, for example, made with a magnesium oxide stabilized transformation toughened zirconia (MgO-TTZ), can be the implant prosthesis itself, notably for a load-bearing joint.

### BACKGROUND TO THE INVENTION

Control of infection, especially following surgery, to include orthopedic implant surgery, is a significant problem. Such things as the sterilization of implanted devices and surgical tools and the administration of drugs such as antibiotics are routinely employed so as to attempt to combat the problem. Recalcitrant or long-lasting infections, however, do occur after such surgery, and they can be difficult to reduce or eliminate.

It would be desirable to ameliorate if not solve the aforesaid problem.

### A MORE FULL DISCLOSURE OF THE INVENTION

Provided hereby is a method for amelioration, control or elimination of bacterial growth or infection, especially with respect to an implant and/or tissue about the implant, which comprises identifying a potential for or a presence of bacterial growth or infection with respect to an implant and/or tissue about the implant proposed for implantation; selecting as the implant a ceramic implant; and implanting the ceramic implant under conditions such that bacterial growth or infection, especially with respect to the implanted ceramic implant and/or the tissue about the implanted ceramic implant, is ameliorated, controlled or eliminated at least in part from the presence of the implanted ceramic implant. The ceramic implant may be a prosthesis, which may be a load-bearing joint replacement implant or implant component.

The invention is useful in medicine and surgery, especially in orthopedics.

Significantly, by the invention, the art is advanced in kind, and the medical practitioner is provided with and directed to an effective infection control agent, a ceramic implant itself. In a particular embodiment, orthopedic surgeons can now—by selecting a ceramic implant prosthesis over, say, those made predominantly of metal, as may be appropriate to the situation—employ another weapon in their arsenal to combat infection in the patient. More particularly, the ceramic may be provided with a highly smooth surface, which may further enhance the treatment of bacterial growth or infection.

Numerous further advantages attend the invention.

The drawings form part of the specification hereof. With respect to the drawings, which are not necessarily drawn to scale, the following is briefly noted:

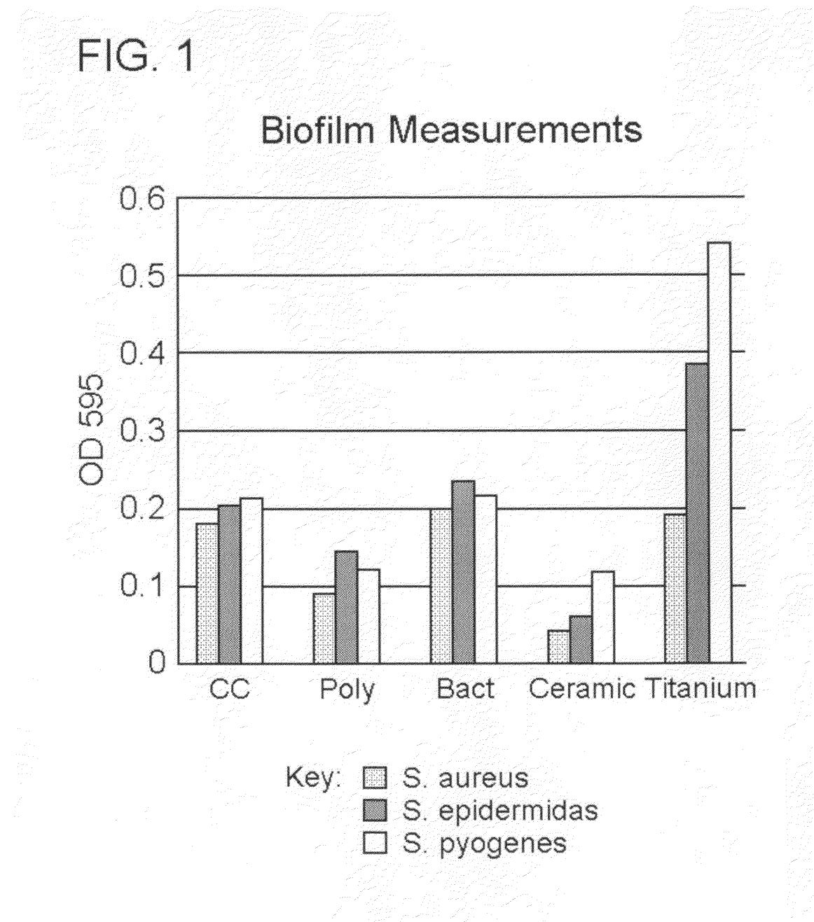

FIG. 1 is a graph of biofilm measurements on various smooth substrates.

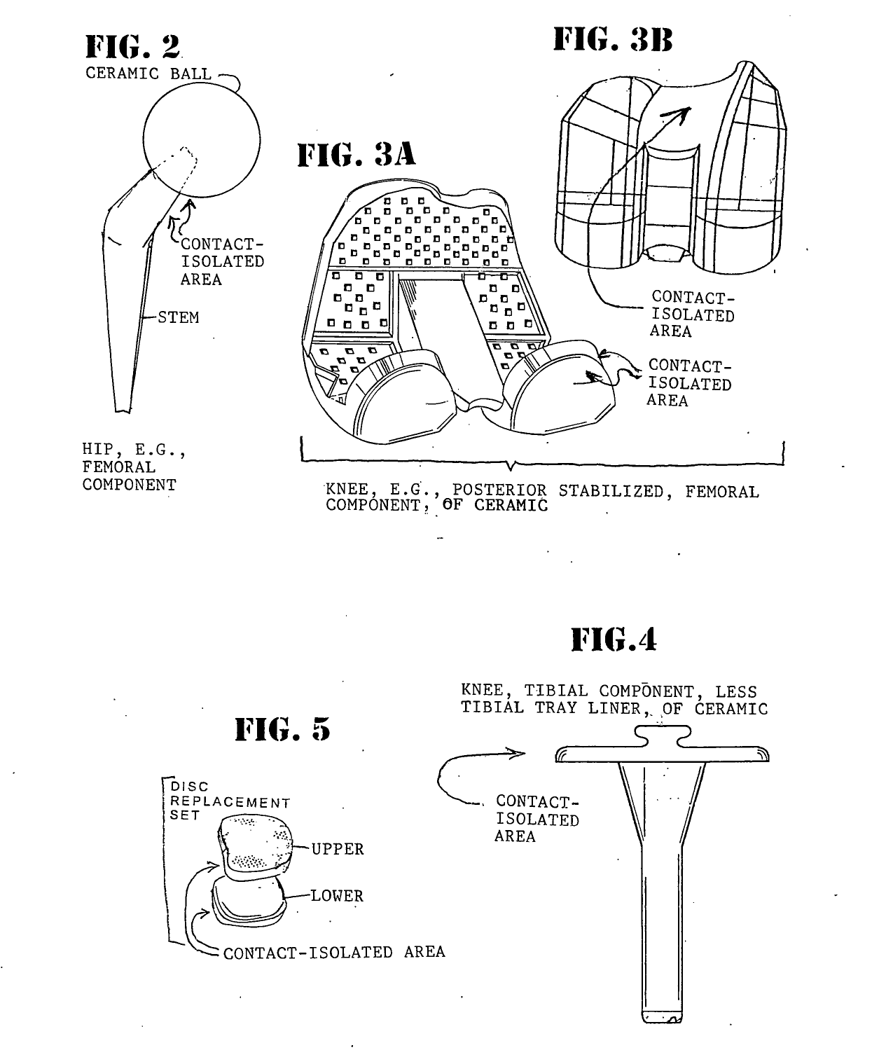

FIGS. 2-5 show various ceramic implant prostheses that may be selected.

The invention can be further understood by the detail set forth below. As with the foregoing, the following is to be taken in an illustrative, not a necessarily limiting, sense.

The ceramic may be any suitable ceramic. The ceramic may come from the family of oxide ceramics, and may be, for instance, a zirconia ceramic, an alumina ceramic, a combined zirconia-alumina ceramic, say, a zirconia-toughened alumina (ZTA) ceramic, and so on. The ceramic can be an MgO-TTZ. See, Pub. No. US 2006/0025866 A1, which is incorporated herein by reference in its entirety, to include its drawings (Mg-TTZ=MgO-TTZ). A smooth surface can be more effective in the amelioration, control or elimination of bacterial growth or infection. The body's natural immunities, drugs such as antibiotics and/or a specialized diet may augment or complete the treatment.

### TEST PROTOCOL AND OUTCOME

The following protocol was employed to test various smooth substances for bacterial growth with respect to S. aureus, S epidermidis, and S. pyogenes:

Substrates were chosen and characterized as follows: cobalt chrome alloy (CC) in disc form, with smoothness of about 2 y inches or less; polyethylene (Poly) in disc form with smoothness of about 20 y inches or less; a supplied, proprietary plastic sample (Bact) in disc form with a smoothness of about 2 y inches or less; MgO-TTZ discs as sliced from a fired rod and not polished (Ceramic); and commercially pure titanium (Titanium) in disc form with smoothness of about 2 y inches or less.

Samples, twelve in number, of each substrate were tested. Bacteria as above were introduced by known concentration from a broth, and allowed to grow in wells at 37° C. for two days.

Results are shown in FIG. 1. This shows the superiority of the ceramic in inhibition of bacterial growth.

The protocol was repeated Substantially the same results ensued, corroborating the superiority of the ceramic in inhibition of bacterial growth.

### FURTHER DISCLOSURE

The ceramic implant prosthesis may be for any suitable part of the body. The ceramic prosthesis may be a load-bearing replacement joint or component therefor, for instance, an enarthrodial joint such as a hip or shoulder; a ginglymous joint such as a knee; a spinal vertebra; and so forth. Compare, FIGS. 2 et seq.

Employment of the ceramic prosthesis may be especially effective in a situation where an implant implanted in the body has areas on it that do not receive physical contact with another solid, for example, an area that is not articulated against in a joint implant, so as to be provided with physical removal or inhibition of bacterial growth such as in effect by wiping with the other solid. In other words, such an area not receiving such physical contact may be termed, “contact-isolated.” See, FIGS. 2 et seq. Such contact-isolated areas, in general, can provide for higher bacterial growth than areas where there is physical contact. In such and in various other situations, for example, in a bone-interfacing situation, where infection otherwise may be difficult to treat, a drug regimen may be employed in conjunction with employment of the present invention. In the bone-interfacing situation, for example, a bone vascular system may assist in delivery of natural bodily immunities, the drug, benefits from a specialized diet, and so forth.

### CONCLUSION TO THE INVENTION

The present invention is thus and hereby provided. Various feature(s), part(s), step(s), subcombination(s) and/or combination(s) can be employed with other feature(s), part(s), step(s), subcombination(s) and/or combination(s) in the practice of the invention, and numerous and sundry adaptations and modifications can be effected within its spirit, the literal claim scope of which is particularly pointed out as follows:

What is claimed is:

1. A method for amelioration, control or elimination of bacterial growth or infection, especially with respect to an implant and/or tissue about the implant, which comprises identifying a potential for or a presence of bacterial growth or infection with respect to an implant and/or tissue about the implant proposed for implantation; selecting as the implant a ceramic implant; and implanting the ceramic implant under conditions such that bacterial growth or infection, especially with respect to the implanted ceramic implant and/or the tissue about the implanted ceramic implant, is ameliorated, controlled or eliminated at least in part from the presence of the implanted ceramic implant.

2. The method of claim 1, wherein a contact-isolated area is identified on the ceramic implant.

3. The method of claim 2, wherein the ceramic implant is a load-bearing orthopedic prosthetic implant.

4. The method of claim 2, wherein the ceramic implant is selected from the group consisting of an enarthrodial joint component, a ginglymous joint component, and a spinal vertebrae component.

5. The method of claim 2, wherein the ceramic implant is selected from the group consisting of a joint ball head, a femoral component for a knee, a tibial component for a knee, and a disc replacement set.

6. The method of claim 1, wherein the ceramic is an MgO-TTZ.

7. The method of claim 2, wherein the ceramic is an MgO-TTZ.

8. The method of claim 3, wherein the ceramic is an MgO-TTZ.

9. The method of claim 4, wherein the ceramic is an MgO-TTZ.

10. The method of claim 5, wherein the ceramic is an MgO-TTZ.
